(19) United States
(12) Patent Application Publication
(10) Pub. No.: US 2019/0100785 A1
(43) Pub. Date: Apr. 4, 2019

(54) METHOD OF PRODUCING UNDENATURED COLLAGEN FROM CARTILAGE WITH LOW TEMPERATURE HYDROLYSIS

(71) Applicant: Robert den Hoed, Sioux Center, IA (US)

(72) Inventor: Robert den Hoed, Sioux Center, IA (US)

(21) Appl. No.: 16/036,368

(22) Filed: Jul. 16, 2018

Related U.S. Application Data
(60) Provisional application No. 62/567,283, filed on Oct. 3, 2017.

(51) Int. Cl. C12P 21/06; C07K 14/78

(52) U.S. Cl. CPC C12P 21/06; C07K 14/78

(57) ABSTRACT

A method of producing undenatured collagen from cartilage uses a low temperature hydrolysis. To begin, cartilage, citric/lactic acid, and water are added to a digestion vessel and stirred. Next, a base and acid stable protease are added and the mixture is stirred again before being set aside. Bones and fat are removed leaving a liquid mixture to which a base is added. Sediment is then filtered, removed, and dried from the liquid mixture to form a collagen powder.

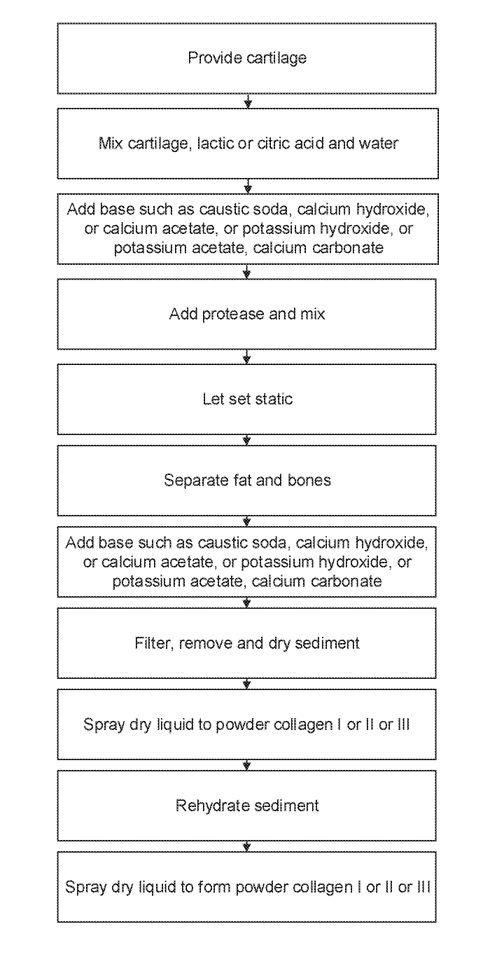

### CROSS REFERENCE TO RELATED APPLICATIONS

This application claims the benefit of U.S. Provisional Application No. 62/567,283 filed Oct. 3, 2017, the contents of this application is hereby incorporated by reference in its entirety.

### BACKGROUND OF THE INVENTION

This invention is directed to a method of producing collagen from cartilage and more particularly to producing collagen from low temperature hydrolysis.

Cartilage is well known as a source of valuable bioactive materials, including collagen, that have widespread applications in medical, health and cosmetic industries for joint and wound healing and skin care. To date, a major drawback to their use has been the difficulty in solubilizing these materials in a collagen non-gelatin stable and active pure form at an industrial scale so that high yield is achieved in an economic manner.

An objective of the present invention is to provide a method of producing collagen from cartilage using low temperature hydrolysis.

Another objective of the present invention is to provide a method of making collagen from cartilage in a collagen non-gelatin stable and pure form at an industrial scale.

These and other objectives will be apparent to those having ordinary skill in the art based upon the following written description, drawings, and claims.

### SUMMARY OF THE INVENTION

A method of making undenatured collagen from cartilage using a low temperature hydrolysis begins by adding cartilage, acid (citric or lactic), and water to a digestion vessel to form a thick slurry like mixture. The mixture is then stirred for about twelve hours at a temperature between 10° and 43° C.

Next, a base and an acid stable protease are added to, in part, adjust the pH, and the mixture is stirred again for between 2 and 24 hours at a temperature between 10° and 43° C. Once stirred the mixture is set aside for 20 minutes to 6 hours.

After the mixture has set the bones and fat are removed from the liquid mixture. A base is then added to adjust the pH level of the liquid mixture. Next, sediment is filtered off, removed, and dried from the liquid mixture to form a collagen powder. The liquid mixture is spray dried to form a collagen powder or further processed by adding papain and bacteria neutrolase, heating the liquid mixture and then spray drying to form a powdered collagen.

### BRIEF DESCRIPTION OF THE DRAWINGS

The FIGURE is a flow diagram of a method of making collagen from cartilage.

### DETAILED DESCRIPTION OF THE PREFERRED EMBODIMENT

A method of making hydrolyzed collagen begins by adding cartilage to a digestion vessel. The cartilage can be from marine, avian, bovine, porcine, or ovine such as trachea, rack cartilage, chicken feet, chicken keel cartilage, marine head and spine cartilage or scapula cartilage. Preferably, 25-500 grams of cartilage is added to the digestion vessel.

Also added to the digestion vessel is lactic acid or citric acid and water. Preferably 22.7 ml of lactic acid or citric acid, or 0.1% to 7% volume, is added and 300-500 mLs of water to form a mixture that is of a substantially thick slurry consistency. Preferably, in terms of common liquid-thickness levels, the slurry should range between a pudding-like thickness to a honey-like thickness. For this arrangement, a thicker slurry consistency is preferable over more thin consistencies and a very thick consistency is preferable over a thick consistency. The mixture is then stirred or mixed within the digestion vessel. Preferably the mixture is stirred for about twelve hours at a temperature of 10°-43° C.

After about twelve hours, caustic soda, calcium hydroxide, calcium acetate, calcium carbonate, potassium hydroxide or potassium acetate is added to the mixture to adjust the pH to 3.2 to 3.3. Next, an acid stable protease is added to the mixture. Preferably, 0.5-3 grams of acid stable protease is added. Once added, the mixture is stirred at a temperature of 10°-43° C. for about 2 to 24 hours.

Next, the mixture is set aside to set for 20 minutes to 6 hours. After the mixture has set, the bones and fat are separated from the liquid of the mixture. Once separated, a base such as caustic soda, calcium hydroxide, calcium acetate, calcium carbonate, potassium hydroxide or potassium acetate are added to the liquid mixture preferably to bring the pH to 5-7.5. Sediment is then filtered off and removed and dried at 100° to 200° F. to form a powdered collagen type I, II, or III. The liquid is spray dried or further processed by adding papain and a bacteria neutrolase such as Enzeco® alkaline protease L-660 or divalent metalloendopeptidase such as PROTEX™ 14L. The liquid is then heated to between 30° to 85° C. for between three to four hours. Finally, the heated liquid is spray dried to form powdered collagen type I, or II, or III.

In one example, the method produced collagen having the following characteristics:

| Substance | Level/Amount | |
|---|---|---|
| Tested/Detected | (Dry Weight Basis/100 g) | |
| Type I | 6.02 | g |
| Type II | 93.30 | g |
| Type III | 0.68 | g |
| Calcium | 18.88 | mg |
| Glucosamine | 30.03 | mg |

| | |
|---|---|
| Chondroitin | 22.31% |
| Hyaluronic Acid | 14.16% |
| Mucopolysaccharides | 50.22% |

What is claimed is:

1. A method of producing collagen from cartilage, comprising the steps of:
adding cartilage to a digestion vessel;
adding water and lactic acid and/or citric acid to the digestion vessel to form a mixture;
stirring the mixture;
adding a base to the mixture;
adding an acid stable protease to the mixture;
stirring the mixture;
setting the mixture aside for a period of time;
separating the bones and fat from the liquid in the mixture;
adding a base to the liquid mixture;
filtering off sediment from the liquid mixture; and
removing and drying the sediment to form a powdered collagen.

2. The method of claim 1 wherein after the sediment is removed the liquid is spray dried to form a powdered collagen of type I, or II, or III.

3. The method of claim 1 further comprising the steps of adding a papain and bacteria neutrolase to the liquid mixture after the sediment is removed, heating the liquid mixture, and spray drying the heated mixture to form a collagen powder or type I, or II, or III.

4. The method of claim 1 wherein the stirring steps are conducted at a temperature between 10° and 43° C.
